(19) United States
(12) Patent Application Publication
(10) Pub. No.: US 2005/0201991 A1
(43) Pub. Date: Sep. 15, 2005

(54) Stem cell culture

(76) Inventors: Peter Andrews, Sheffield (GB); Jon Draper, Sheffield (GB)

Correspondence Address: KLARQUIST SPARKMAN, LLP, 121 SW SALMON STREET, SUITE 1600, PORTLAND, OR, 97204, US

(21) Appl. No.: 10/507,884

(22) PCT Filed: Mar. 18, 2003
(86) PCT No.: PCT/GB03/01111

§ 371 Date: May 9, 2005

(30) Foreign Application Priority Data
Mar. 19, 2002 (GB) 0206422.8
May 8, 2002 (GB) 0210458.6

(51) Int. Cl. A61K 48/00; C12N 15/09; C12N 5/08; C12N 15/85

(52) U.S. Cl. 424/93.21; 435/320.1; 435/455; 435/325; 435/366

(57) ABSTRACT

We describe a method to manipulate the phenotype of stem cells, preferably embryonic stem cells (ES), including nucleic acids and vectors used in said methods.

The invention relates to a method to manipulate the phenotype of stem cells, preferably embryonic stem cells (ES), including nucleic acids and vectors used in said methods.

During mammalian development those cells that form part of the embryo up until the formation of the blastocyst are said to be totipotent (e.g. each cell has the developmental potential to form a complete embryo and all the cells required to support the growth and development of said embryo). During the formation of the blastocyst, the cells that comprise the inner cell mass are said to be pluripotential (e.g. each cell has the developmental potential to form a variety of tissues).

Embryonic stem cells (ES cells, those with pluripotentiality) may be principally derived from two embryonic sources. Cells isolated from the inner cell mass are termed embryonic stem (ES) cells. In the laboratory mouse, similar cells can be derived from the culture of primordial germ cells isolated from the mesenteries or genital ridges of days 8.5-12.5 post coitum embryos. These would ultimately differentiate into germ cells and are referred to as embryonic germ cells (EG cells). Each of these types of pluripotential cell has a similar developmental potential with respect to differentiation into alternate cell types, but possible differences in behaviour (eg with respect to imprinting) have led to these cells to be distinguished from one another.

Typically ES/EG cell cultures have well defined characteristics. These include, but are not limited to; maintenance in culture for at least 20 passages when maintained on fibroblast feeder layers; produce clusters of cells in culture referred to as embryoid bodies; ability to differentiate into multiple cell types in monolayer culture; and express ES/EG cell specific markers.

Until very recently, in vitro culture of human ES/EG cells was not possible. The first indication that conditions may be determined which could allow the establishment of human ES/EG cells in culture is described in WO96/22362. The application describes cell lines and growth conditions which allow the continuous proliferation of primate ES cells which exhibit a range of characteristics or markers which are associated with stem cells having pluripotent characteristics.

More recently Thomson et al. (1998) have published conditions in which human ES cells can be established in culture. The above characteristics shown by primate ES cells are also shown by the human ES cell lines. In addition the human cell lines show high levels of telomerase activity, a characteristic of cells which have the ability to divide continuously in culture in an undifferentiated state. Another group (Reubinoff et. al., 2000) have also reported the derivation of human ES cells from human blastocyts. Shamblott et. al., 1998 have also described EG cell derivation. In Lake et al J Cell Science 2000, 113:555-66 and Rathjen et al J Cell Science 1999, 112: 601-12, ectodermal stem cells are disclosed. The above references are each both incorporated by reference in their entirety.

A problem associated with the maintenance of ES/EG cells in culture is that it is extremely difficult to maintain a pure population of proliferating ES/EG cells. ES/EG cells require the presence of fibroblast feeder layers in order to retain their ability to divide in an undifferentiated state for several generations. If the feeder layers are removed then the cells differentiate. The differentiation is often to neurones or muscle cells but the exact mechanism by which this occurs and its control remains unsolved. A further problem is that even when grown under optimal conditions, the cells tend to spontaneously differentiate leading to cultures in which undifferentiated cells are admixed with variable numbers of their differentiated derivatives.

Fibroblast growth factor-4 (FGF-4), also referred to as heparin secretory transforming protein 1 (HST-1) or Kaposi sarcoma oncogene, is a member of the fibroblast growth factor family. FGF-4 has been shown to be a signalling molecule involved in growth and differentiation via the dimerisation of its tyrosine kinase receptors. In vivo, the FGF-4 gene is expressed in the blastocyst inner cell mass and in specific embryonic tissues, but is transcriptionally silent in the adult (Yuan et al., 1995). Likewise, in culture, FGF-4 expression has also been shown to be restricted to undifferentiated embryonic stem cells and embryonal carcinoma (EC) cell lines (Yuan et al., 1995). Expression of FGF4 is controlled by cis-regulatory elements located in the 5′-flanking region and by a powerful enhancer located approximately 3 kb downstream from the transcription start site (Lamb et al., 1996). Repression of FGF-4 gene transcription occurs in differentiated cells due to a reduction in the ability of nuclear factors to bind to an octamer motif and an adjacent High Mobility Group motif within the enhancer.

Rex-1 (Zfp-42 gene) is a developmentally regulated acidic zinc finger gene. Rex-1 mRNA demonstrates a similar expression profile to FGF-4 and is detected in undifferentiated embryonic stem and EC cells, mouse embryos at the blastocyst stage, and also in the trophectoderm and meiotic germ cells of the adult mouse testis (Rogers et al., 1991). Transcription of Rex-1 is reduced during retinoic acid-induced differentiation (Hosler et al., 1993). An octomer motif (ATTTGCAT) at position −220 in the murine Rex-1 promoter has been shown to be required for promoter activity in mouse F9 teratocarcinoma cells (Hosler et al., 1993). This octamer motif is a binding site for members of the POU (Pit-Oct-Unc) domain family of DNA-binding proteins (Anderson & Rosenfeld, 2001), including Oct-3/4 and its alternatively spliced forms Oct-5 and-6, all expressed by EC and ES cells (Schöler 1991). Oct-3 has been shown to bind to the ATGCAAAT motif in Rex-1 (Ben-Shushan et al., 1998) and FGF-4 (Ambrosetti et al., 1997).

Selectable markers can be used to select a required genotype. These markers confer a new phenotype, often based on resistance to antibiotics such as ampillicin, (Sykes & Matthew, 1976) tetracycline (Franklin, 1967), chloramphenicol (Le Grice & Matzura, 1981), kanamycin (neomycin), hygromycin and puromycin (Karreman et al., 1998). However, the drug concentrations required to reduce the survival of cells carrying “unwanted” genotypes may have a long-term effect on the cells, for example, levels have been shown to inhibit progenitor cell proliferation and/or differentiation in hematopoietic stem cells (Chinswangwatanakul et al., 1998). An alternative strategy for selection of living cells is based on the green fluorescent protein (GFP) from the jellyfish Aequorea victoria, and variants thereof, including blue-(BFP), cyan-(CFP) and yellow(YFP)-fluorescent protein (Beavis et al., 1999) and also red fluorescent protein (RFP) from Discosoma coral (Matz et al., 1999).

Suicide gene therapy is one of several gene therapeutic approaches to treat disease, particularly cancer. A suicide gene is a gene, usually of viral or prokaryotic origin that encodes a protein, typically an enzyme, that in itself is non-toxic to the genetically modified cell. However, when a cell is exposed to a specific non-toxic prodrug, this is selectively converted by the gene product into toxic metabolites that kill the cell (See Table 1). The suicide gene most commonly employed, both in experimental and clinical settings, is herpes simplex thymidine kinase (HSVtk). For example a c-erb-2 promoter-mediated expression of HSVtk has been shown to confer selective cytotoxicity of human breast cancer cells to gancyclovir (Maeda et al., 2001). Other examples of suicide genes are those which express products that are directly toxic to the cell, such as the diptheria toxin or pseudomonas exotoxin or which inhibit protein synthesis (Dilber & Gabrton, 2001).

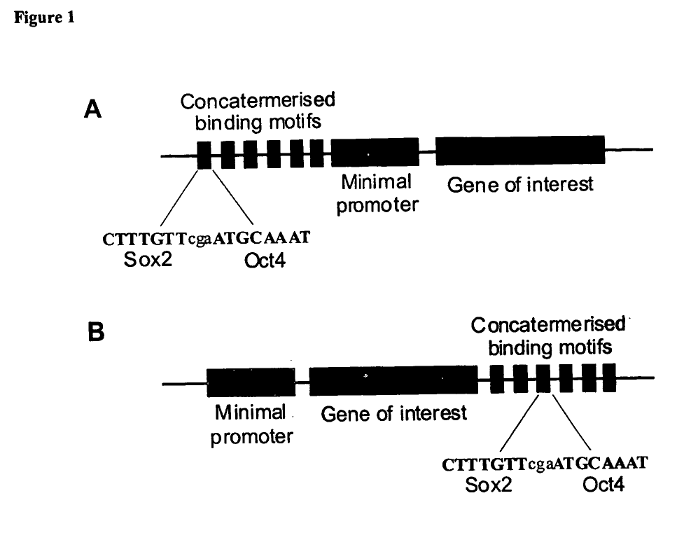

We have now examined expression of FGF4 in stock cultures of human ES cells. If these cells are sorted for expression of the surface antigen marker SSEA3 characteristically expressed by undifferentiated human ES cells, (Thomson et al 1998; Draper et al 2002), we have found expression of FGF4 exclusively in the SSEA3-positive cells, and not in the SSEA3-negative cells that constitute their spontaneous differentiated derivatives (FIG. 1). By contrast, the SSEA3-negative cells expressed predominantly or exclusively other marker genes characteristic of differentiated derivatives—e.g. AFP and HBZ (yolk sac and embryonic hematopoietic cells), HCG (trophectoderm), ND1, Sox1 (neural cells).

We have determined that the inclusion of a motif from the FGF4 enhancer in a promoter, for example, the thymidine kinase promoter, confers stem cell specific expression on reporter genes under its control. We have exploited this discovery to provide a cell culture system which facilitates the maintenance of stem cells, particularly embryonic stem cells, in an undifferentiated state. We also provide differentiated cells/tissues the genome of which includes a nucleic acid construct comprising a promoter which has substantially a stem cell specific expression pattern which controls expression of a gene the expression of which allows the selective ablation of cells which have de-differentiated to a stem cell phenotype thereby allowing their removal from a population of differentiated cells. The invention therefore relates, amongst other things, to the provision of methods to manipulate the phenotype of stem cells, preferably embryonic stem cells (ES).

According to an aspect of the invention there is provided a method to manipulate stem cell phenotype comprising;
(i) providing a stem cell which has been transfected with a nucleic acid molecule wherein said molecule includes a promoter which comprises at least one nucleic acid sequence motif which confers substantially stem cell specific expression on at least one selectable marker gene;
(ii) providing conditions conducive to the proliferation of said cell in (i) above; and optionally
(iii) maintaining and/or storing said cell.

In a preferred method of the invention said conditions are in vitro cell culture conditions.

In a further preferred method of the invention said promoter is a synthetic promoter. A synthetic promoter maybe a minimal promoter which includes motifs which direct stem cell specific expression with a core promoter (e.g. heatshock promoters HSP68, thymidine kinase promoter).

In a preferred method of the invention, the motif binds the transcription factors of the POU domain family, more specifically Oct-3/4 and its alternatively spliced forms Oct-5 and -6.

In a further preferred method of the invention, the motif is derived from the FGF-4 promoter, preferably of human origin. Preferably said motif is at least one copy of the sequence CTTTGTT and ATGCAAAT; or CTTTGTT and ATGCTAAT.

In a preferred method of the invention said motif comprises at least one copy of the motif CTTTGTTXATGCAAAT wherein X is at least one nucleotide base which serves as a linking molecule.

In an alternative method of the invention said motif comprises at least one copy of the motif CTTTGTTXATGCIAAT wherein X is at least one nucleotide base which serves as a linking molecule.

An example of such a motif is CTTTGTTcgaATGCAAAT.

In a further preferred method of the invention said nucleic acid molecule comprises a plurality of motifs according to the invention. Preferably there is provided at least 2 motifs. More preferably still there is provided at least 3, 4, 5, 6, 7, 8, 9 or 10 motifs. In a further preferred method of the invention the motif is derived from the Rex-1 (zfp-42) promoter, preferably Rex-1 is of human origin.

In an alternative preferred method of the invention the motif is derived from the murine Rex-1 promoter, for example, ATTTGCAT.

In a further preferred method of the invention stem cells are selected from a group consisting: haemopoietic stem cells; neural stem cells; bone stem cells; muscle stem cells; mesenchymal stem cells; trophoblastic stem cells; epithelial stem cells (derived from organs such as the skin, gastrointestinal mucosa, kidney, bladder, mammary glands, uterus, prostate and endocrine glands such as the pituitary); endodermal stem cells (derived from organs such as the liver, pancreas, lung and blood vessels); embryonic stem (ES) cells; embryonal germ (EG) cells. Preferably said stem cells are primate, preferably human.

In an alternative further preferred method of the invention said stem cells are embryonal carcinoma cells. Preferably said embryonal carcinoma cells are TERA2 cells. Ideally said embyonal carcinoma cells are NTERA 2 cells.

In a yet further preferred method of the invention said selectable marker is a gene encoding a polypeptide capable of fluorescence emission when excited by light. More preferably still said polypeptide is selected from the group consisting of; BFP, CFP, YFP, GFP, RFP and variants thereof.

GFP of the jelly fish Aequorea victoria has an excitation maximum 395 nm, an emission at 510 nm and does not require the addition of an exogenous factor. Mutant forms of GFP are also known with altered fluorescence emission properties, see WO9821355; U.S. Pat. Nos. 5,804,387; 5,777,079; and 5,625,048, which are incorporated by reference.

In an alternative preferred method of the invention said selectable marker is a gene encoding a polypeptide which confers resistance to antibiotics selected from the group consisting of; β lactamase, tetracycline resistance polypeptide, chloramphenicol acetyltransferase, aminoglycoside phosphotransferase, hygromycin phosphotransferase; puromycin N acetyltransferase, or variants thereof.

According to a further aspect of the invention there is provided an isolated nucleic acid molecule comprising a promoter of a gene which has substantially stem cell specific expression operably linked to at least one selectable marker.

In a preferred embodiment of the invention said promoter comprises a motif which confers substantially stem cell expression of said selectable marker. Preferably said motif is bound by a transcription factor of the POU family of transcription factors, for example Oct-3/4 and its alternatively spliced forms Oct-5 and-6.

Preferably said motif is at least one copy of the sequence CTTTGTT and ATGCAAAT; or CTTTGTT and ATGCTAAT.

In a preferred method of the invention said motif comprises at least one copy of the motif CTTTGTTXATGCAAAT wherein X is at least one nucleotide base which serves as a linking molecule.

In an alternative method of the invention said motif comprises at least one copy of the motif CTTTGTTXATGCTAAT wherein X is at least one nucleotide base which serves as a linking molecule.

In an alternative preferred embodiment said motif is derived from the Rex-1 (zfp-42) promoter, preferably human Rex-1 (zfp-42) or murine Rex-1.

In a further preferred embodiment of the invention said selectable marker encodes a prodrug activating polypeptide.

In a preferred embodiment of the invention said prodrug activating polypeptide is selected from the group consisting of: thymidine kinase; triphosphate cytosine deaminase; 5 guanosine -xanthine phosphoribosyl; transferase; purine nucleoside phosphorylase; nitroreductase; CYP 2B1 (cytochrome P450); CYP 4B1 (cytochome P450); Cytochrome P450; Varicella zoster virus thymidinekinase (VZVtk); β-glucosidase; β-lactamase; b-glucoronidase; Carboxylesterase; Alkaline phosphatase; carboxypeptidase G2, or variants thereof.

According to a further aspect of the invention there is provided a vector comprising a nucleic acid molecule according to the invention.

Typically the vector is viral based. Preferred vectors are derived from the adenoviral, adeno-associated viral or retroviral genomes. The vectors can be derived from the human adenovirus genome. Particularly preferred vectors are derived from the human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve i particular expression characteristics or permit repeat administration or lower immune response are preferred. Most preferred are human adenoviral type 5.

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein an gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al., U.S. Pat. No. 5,871,726 issued Feb. 16, 1999 the entire teachings of which are herein incorporated by reference.

The viruses may also be designed to be selectively replicating viruses. Particularly preferred selectively replicating viruses are described in Ramachandra, et al. PCT International Publication No. WO 00/22137, International Application No. PCT/US99/21452 published Apr. 20, 2000 and Howe, J., PCT International Publication No. WO 00/22136, International Application No. PCT/US99/21451 published Apr. 20, 2000.

It has been demonstrated that viruses which are attenuated for replication are also useful in the therapeutic arena. For example the adenovirus d11520 containing a specific deletion in the E1bS5K gene (Barker and Berk (1987) Virology 156: 107) has been used with therapeutic effect in human beings. Such vectors are also described in McCormick (U.S. Pat. No. 5,677,178 issued Oct. 14, 1997) and McCormick, U.S. Pat. No 5,846,945 issued Dec. 8, 1998.

According to a further aspect of the invention there is provided a stem cell transfected with a DNA molecule or a vector according to the invention.

In a preferred embodiment of the invention said cell is stably transfected. Alternatively said cell is transiently transfected.

In a preferred embodiment of the invention said stem cell is selected from the group consisting of: haemopoietic stem cells; neural stem cells; bone stem cells; muscle stem cells; mesenchymal stem cells; trophoblastic stem cells; epitbelial stem cells (derived from organs such as the skin, gastrointestinal mucosa, kidney, bladder, mammary glands, uterus, prostate and endocrine glands such as the pituitary); endodermal stem cells (derived from organs such as the liver, pancreas, lung and blood vessels).

Preferably said cell is an embryonic stem cell. Alternatively said cell is an embryonal carcinoma cell. Preferably said embryonal carcinoma cells are TERA2 cells. Ideally said embyonal carcinoma cells are NTERA 2 cells.

According to a further aspect of the invention there is provided a differentiated cell transfected with a DNA molecule or vector according to the invention.

In a preferred embodiment of the invention said differentiated cell is selected from the group consisting of: a nerve cell; a mesenchymal cell; a muscle cell (cardiomyocyte); a liver cell; a kidney cell; a blood cell (eg erythrocyte, CD4+ lymphocyte, CD8+ lymphocyte; panceatic β cell; epithelial cell (eg lung, gastric,); and a endothelial cell.

Conventional methods to introduce DNA into cells are well known in the art and typically involve the use of chemical reagents, cationic lipids or physical methods.

Chemical methods which facilitate the uptake of DNA by cells include the use of DEAE-Dextran (Valieri and Pagano Science 175: p434). DEAE-dextran is a negatively charged cation which associates and introduces the DNA into cells but which can result in loss of cell viability. Calcium phosphate is also a commonly used chemical agent which when co-precipitated with DNA introduces the DNA into cells (Graham et al Virology (1973) 52: p456).

The use of cationic lipids (eg liposomes (Felgner (1987) Proc. Natl. Acad. Sci USA, 84:p7413) has become a common method since it does not have the degree of toxicity shown by the above described chemical methods. The cationic head of the lipid associates with the negatively charged nucleic acid backbone of the DNA to be introduced. The lipid/DNA complex associates with the cell membrane and fuses with the cell to introduce the associated DNA into the cell. Liposome mediated DNA transfer has several advantages over existing methods. For example, cells which are recalcitrant to traditional chemical methods are more easily transfected using liposome mediated transfer.

More recently, other non-liposome based chemical transfectant agents have become available, for example ExGen500 (polyethylenimine), produced by MBI Fermentas. ExGen500 is particularly effective for transfection of human ES cells (Eiges, 2001).

More recently still, physical methods to introduce DNA have become effective means to reproducibly transfect cells. Direct microinjection is one such method which can deliver DNA directly to the nucleus of a cell (Capecchi (1980) Cell, 22:p479). This allows the analysis of single cell transfectants. So called “biolistic” methods physically shoot DNA into cells and/or organelles using a particle gun (Neumann (1982) EMBO J, 1: p841). Electroporation is arguably the most popular method to transfect DNA. The method involves the use of a high voltage electrical charge to momentarily permeabilise cell membranes making them permeable to macromolecular complexes. However physical methods to introduce DNA do result in considerable loss of cell viability due to intracellular damage. These methods therefore require extensive optimisation and also require expensive equipment.

More recently still a method termed immunoporation has become a recognised techinque for the introduction of nucleic acid into cells, see Bildirici et al Nature (2000) 405, p298. The technique involves the use of beads coated with an antibody to a specific receptor. The transfection mixture includes nucleic acid, typically vector DNA, antibody coated beads and cells expressing a specific cell surface receptor. The coated beads bind the cell surface receptor and when a shear force is applied to the cells the beads are stripped from the cell surface. During bead removal a transient hole is created through which nucleic acid and/or other biological molecules can enter. Transfection efficiency of between 40-50% is achievable depending on the nucleic acid used. The above described methods for the introduction of DNA into cells are incorporated by reference.

According to a further aspect of the invention there is provided a cell culture comprising a transfected stem cell according to the invention.

According to a yet further aspect of the invention there is provided a cell culture comprising a differentiated cell according to the invention.

According to a yet further aspect of the invention there is provided at least one organ/tissue comprising at least one differentiated cell according to the invention.

According to a yet further aspect of the invention there is provided a method to ablate a cell according to the invention comprising exposing said cell to an agent to which said cell has been sensitized.

In a preferred method of the invention said cell is a differentiated cell which has or is de-differentiating to a stem cell or lineage restricted stem cell.

In a further preferred method of the invention said method is an in vivo method.

In an alternative preferred method of the invention said method is an in vitro method.

In a further preferred method said agent is selected from the group consisting of: ganciclovir; 5-fluorouracil (5-FU); 6-Tg triphosphate; 6-methylpurine; hydroxylamine; 4-hydroperoxycyclo-phosphoainide; DNA-alkylating; alkylating metabolites; araM-MP; cyanide; vinca alk; phenolmustard; SN-38; phenolmustard; etoposide; benzoic acid mustards.

In an alternative preferred method of the invention said agent is diptheria toxin or pseudomonas exotoxin.

An embodiment of the invention will now be described by example only and with reference to the following table and figures:

Table 1 represents a summary of prodrug activating enzymes and their respective prodrugs and activated drugs;

Table 2 illustrates the PCR primers used to detect gene expression in human ES cells;

FIG. 1 is a schematic diagram of FGF4 promoter cassettes illustrating DNA binding motifs; Human/Mouse Sox2 DNA binding motif: CTTTTGTT Human Oct4 DNA binding motif: ATGCAAAT Mouse Oct4 DNA binding motif: ATGCTAAT;

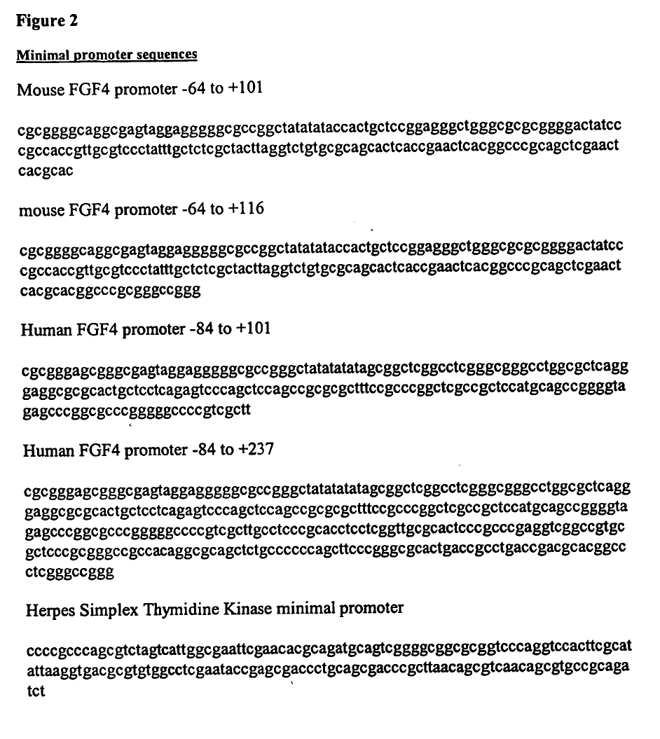

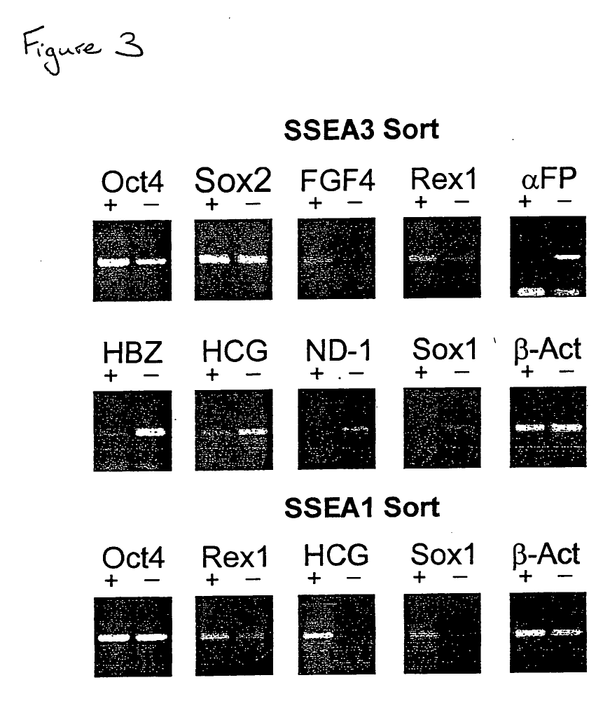

FIG. 2 represents the nucleic acid sequences of minimal human and murine FGF4 promoters;

FIG. 3 shows reverse transcriptase (RT)-PCR analysis of SSEA3(+) and (−), and SSEA1(+) and (−), subsets of H7 human ES cells isolated using fluorescence activated cell sorting. After screening for genomic contamination (data not shown), 1 μg total RNA was reverse transcribed into first-strand cDNA, which was then subject to PCR using primers specific to human Oct4, Sox2, FGF4, Rex1, AFP, HBZ, HCG, NeuroD1 (ND-1), Sox1 and βActin (loading control). Note that especially FGF4 is exclusively expressed in the SSEA3 positive cells which correspond to the undifferentiated cells (Draper et al 2002). By contrast genes typical of differentiated derivatives (e.g. α FP, HB2, HCG, ND-1, SOX1) are exclusively or predominately expressed in the SSEA-3 negative cells;

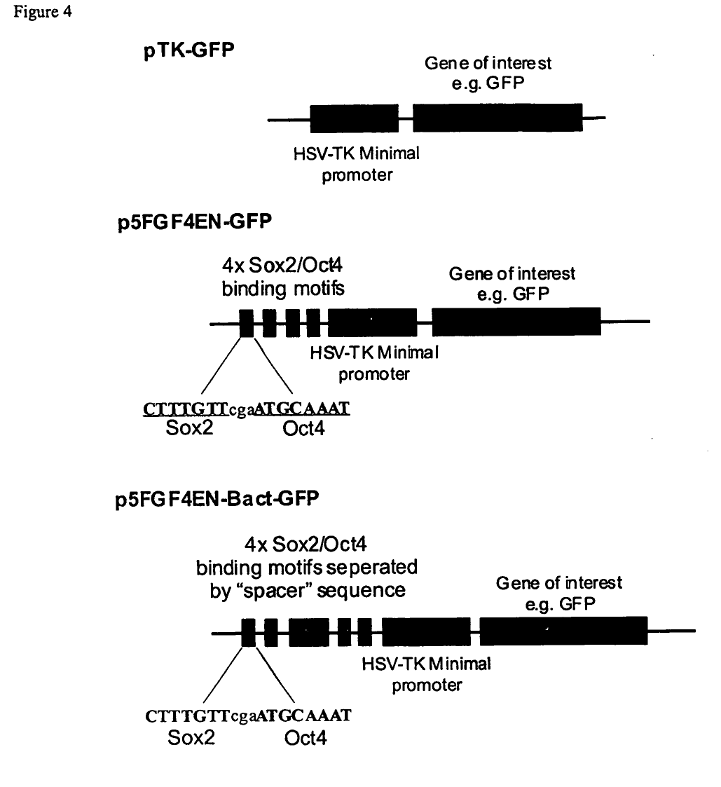

FIG. 4 illustrates examples of GFP expression vector constructs;

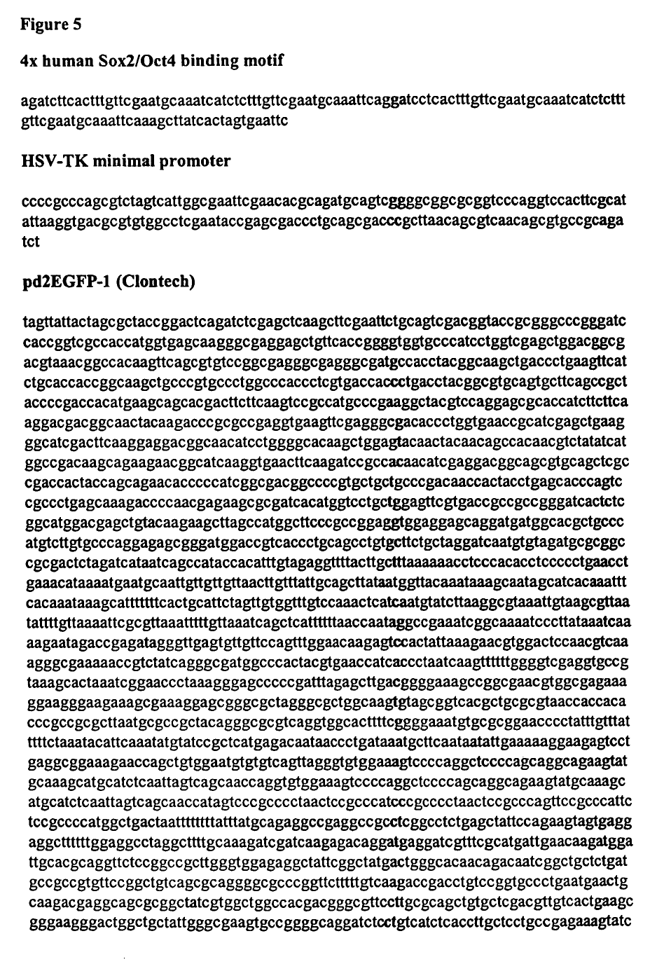

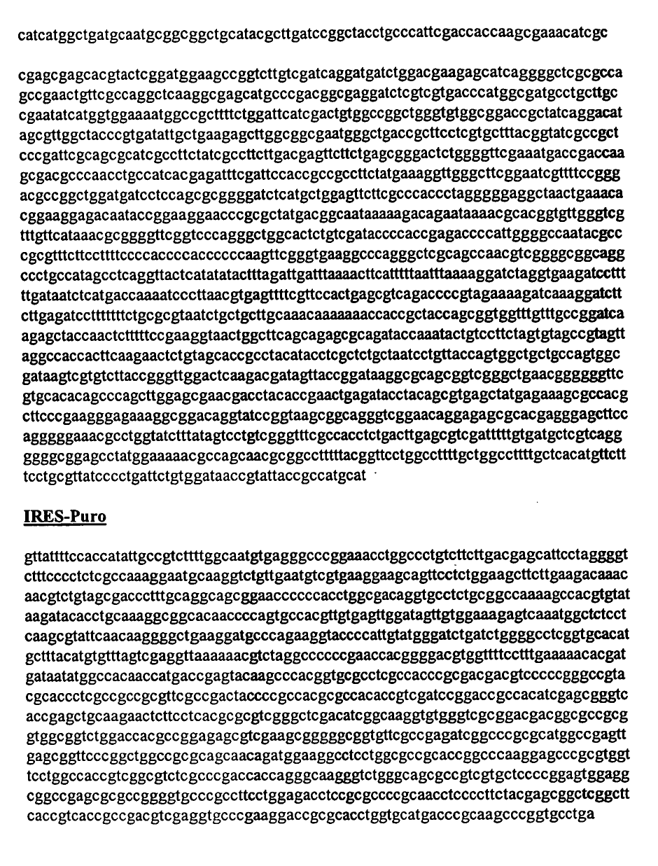

FIG. 5 illustrates the sequence elements of a GFP expression vector construct;

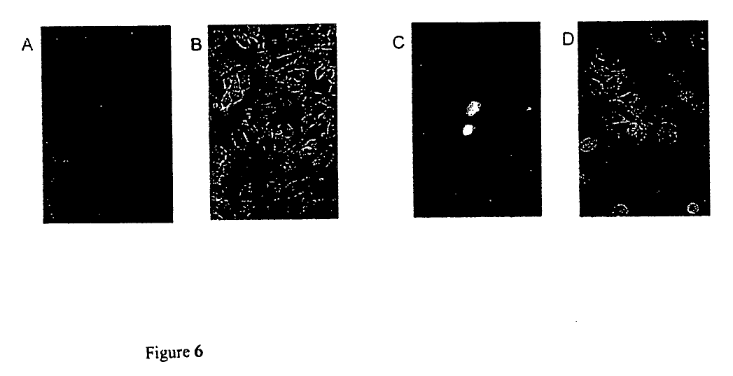

FIG. 6 illustrates transfection of EC/ES cells with GFP expression vectors including FGF4 enhancer motifs. Human EC cell line NTERA2/D1 was transfected with a control plasmid (pTK-GFP) containing only the TK basal promoter and GFP (FIGS. 4A and B—fluorescence and phase respectively). Note the low level basal GFP expression provided by the TK promoter. p5FGF4EN-GFP, a plasmid with four copies of the human Sox2/Oct4 binding motif inserted immediately 5′ to the TK promoter of pTK-GFP, was also transfected into NTERA2/d1 (FIG. 4 C and D). Note the substantially enhanced GFP expression compared to pTK-GFP alone. Similar results were obtained with H7 hES cells transfected with the same pair of vectors;

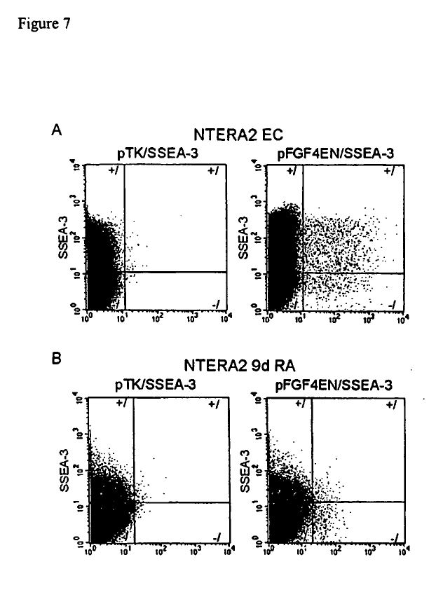

FIG. 7 is a FACS analysis of EC cells transfected with a FGF 4 reporter plasmid. Two fluorochrome flow cytometric analysis using p5FGF4EN-GFPIP reporter combined with SSEA-3 labelling of undifferentiated NTERA2 human EC cells (A) and retenoic acid treated cells (B) demonstrates that GFP expression from FGF-4 reporter is greatly reduced upon differentiation; this reduction is concomittant with a reduction is SSEA-3 expression. The same experiment performed using the pTK-GFPIP control vector which lacks the 4× Oct4/Sox-2 binding motifs is included for comparison; and

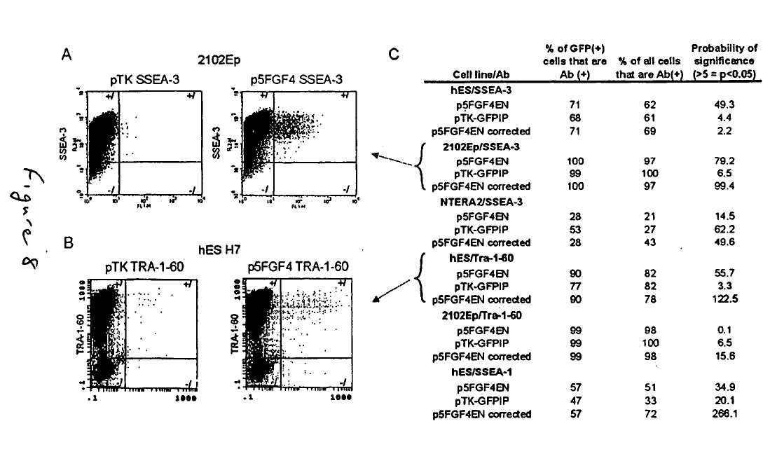

FIG. 8 is a FACS analysis of EC and ES cells transfected with various reporter constructs. Transient transfection of human EC cells (2102EP; A) or ES cells (H7;B) with an FGF-4 enhancer reporter (p5FGF4EN) show that there is a substantial increase in fluorescence when compared to an identical vector lacking the Sox/Oct 4 binding motifs (pTK-GFPIP). Chi squared based statistical analysis of co-labelling of transfected cells with surface markers characteristic of an undifferentiated phenotype (SSEA-3, TRA-1-60) or differentiated derivatives (SSEA-1) indicates that FGF-4 enhancer activity is associated with, for example, TRA-1-60 in human ES cells.

### MATERIALS AND METHODS

GFP Vector Constructs

Three mammalian expression vectors encoding GFP were constructed
1. pTK-GFP: The expression of GFP was driven only by the minimal promoter from the HSV-Tk gene (TK minimal promoter was inserted into the Agel/Apal restriction sites in pd2EGFP-1 (clontech).
2. p5FGF4EN-GFP: The expression of GFP was driven by an enhancer construct incorporating 4 copies of the human FGF4 enhancer inserted 5′ to the HSV-Tk minimal promoter (4× human Sox2/Oct4 DNA binding motif was inserted in the Bglll/EcoRl restriction sites immediately 5′ to the TK promoter of pTK-GFP.

pSFGF4EN-Bact-GFP. This construct is similar to pSFGF4EN-GFP, except that two pairs of 2× human Sox2/Oct4 DNA binding motifs are separated by a 400bp region of β-actin.

Variations of these vectors including pTK-GFP, p5FGF4EN-GFP and p5FGF4EN-Bact-GFP with WES site linking an antibiotic selection marker, for example puromycin or neonmycin resistance, to the expression of GFP were also created (IRESantibiotic selection marker sequences were inserted in the Notl cloning site immediately 3′ to the GFP sequence).

Cell Lines and Culture Conditions

The human embryonal carcinoma (EC) cell line, NTERA2 cl D1, was cultured in DMEM with 10% fetal calf serum as previously described (Andrews et al 1984). Cells were harvested using 0.25% trypsin and 1 mM EDTA in calcium and magnesium free Dulbecco's phosphate buffered saline as previously described (Andrews 1984) and seeded at 6×10⁴ cells per cm² the day before transfection.

The hES cell line H7 and H14 (Thomson et al 1998) were cultured in “Knock-Out” DMEM (GICO Life Technologies Ltd) supplemented with 20% Serum Replacement (GE3CO Life Technologies Ltd) at 37 C under a humidified atmosphere of 5% CO₂ in air, on mitomycin C inactivated mouse embryo fibroblasts (Thomson et al 1998). The cells were harvested by exposure for 3 min at 37 C to a solution of 0.05% trypsin in Calcium and Magnesium free Dulbecco's Phosphate Buffered saline containing 1 mM EDTA. When the cells detached they were resuspended in fresh medium and plated into a fresh tissue culture vessel, precoated with a 1:30 dilution of Matrigel (Becton Dickinson), at 6×10⁴ cells per cm².

Alternatively, for subcultivation, the cells were harvested by treatment with 1 mg/ml collagenase IV, and dispersed by scraping, to maintain the cells in small clumps.

Differentiation was induced by incorporating all-trans-retinoic acid (Eastman-Kodak) (10⁻⁵ M) into the medium as described for human EC cells (Andrews, 1984).

Cell Transfection

Human EC and ES cells were transfected by the same protocol: After incubation at 37° C. overnight the cells were transfected, following the manufacturer's protocol, as follows: 9.5 μg DNA was diluted into 300 μl 0.15 M NaCl and mixed with 21 μl ExGen 500 (MBI Fermentas) and vortexed. After incubating at room temperature for 10 min, the DNA: ExGen solution was mixed with 3 ml culture medium and added a culture of ES cells as described above in a 35 mm (9.6 cm²) dish. After incubating for 6-12 hours at 37 C, the medium was removed and the cells fed with fresh medium and fluorescence observed by microscopy under UV epi-illumination.

Surface Antigen Expression

Cell surface antigen expression of cultured cells was assessed by indirect immunofluorescence detected by flow cytofluorimetry, after harvesting the cultures as single cell suspensions using trypsin-EDTA, as described previously (Andrews et al 1987, Fenderson et al 1987). The following primary monoclonal antibodies were used to detect surface antigen expression: MC631, anti stage specific embryonic antigen 3 (SSEA3) (Shevinsky et al 1982), MC813-70, anti stage specific embryonic antigen 4 (SSEA4) (Kannagi et al 1983), MC480, anti stage specific embryonic antigen 1 (SSEA1) (Solter and Knowles 1978), TRA-1-60 and TRA-1-81 (Andrews et al 1984a), TRA-2-54, anti liver/kidney/bone, alkaline phosphatase (Andrews et al 1984c), TRA-1-85, anti-Ok(a) (Williams et al 1988) and anti-Thy1 (McKenzie and Fabre 1981). FITC-labeled goat anti-mouse IgM or anti-IgG was used as the secondary antibody, as appropriate to the isotype of the primary antibody. In some experiments, cells that were either positive or negative for SSEA3 and SSEA1 were isolated by fluorescence activated cell sorting (Andrews et al 1987).

RT.PCR Analysis of Gene Expression

Total RNA was extracted from ES cells, sorted for antigen expression, using Tri Reagent (Sigma) and was treated with DNase (DNA-Free, Ambion). Subsequently, no genomic contamination could be detected by PCR (data not shown). One microgram of total RNA was used for each reverse transcription (RT) reaction as previously described (Duran et al, 2001). Equal aliquots of RT product were then subjected to PCR using the primers and conditions summarized in Table 1. In all cases 35 PCR cycles were used except for Oct4 (28 cycles), Rex-1 (30 cycles) and β-actin (20 cycles). The PCR primers for AFP were those reported by Schuldiner et al (2000); those for NeuroD1 and β-actin were reported by Duran et al (2001). The remaining primers were designed by using the PRIMERSELECT package from the DNASTAR suite of programs, and the specificity of the RT-PCR products was confirmed by sequencing.

The putative human Rex1 sequence was obtained by searching for ESTs homologous to mouse Rex1 (ZFP-42, NM_{—}009556) using the BLAST protocol (Altschul, et al 1990). One EST (AW665472) mapped to the 3′ end of an open reading frame predicted by Fgenesh analysis (CGG Web Server at http://genomic.sanger.ac.uk) on chromosome 4q35.2 (a region syntenic to mouse Rex1 on chromosome 8). The protein translation of this ORF shows very high homology (unpublished data) with the mouse Rex-1 protein (Ben-Shushan et al 1998). The complete ORF was isolated by PCR from human ES clone H7 cDNA and sequenced, in triplicate. The human Rex1 sequence was submitted to Genbank (AF450454).

### REFERENCES

Anderson B and Rosenfeld M G. POU domain factors in the neuroendocrine system: lessons from developmental biology provides insights into human disease. Endocrine Reviews, 2001;22(l):2-35

Ambrosetti D C, Basilico C and Dailey L. Synergistic activation of the fibroblast growth factor 4 enhancer by Sox2 and Oct-3 depends on protein-protein interactions facilitated by a specific spatial arrangement of factor binding sites. Mol. Cell. Biol. 1997;17:6321-6329.

Beavis A J and Kaletja R F. Simulataneous analysis of the cyan, yellow and green fluorescent proteins by flow cytometry using single-laser excitation at 458nm. Cytometry 1999; 37:68-73

Ben-Shushan E, Thompson J R, Gudas L J and Bergman Y. Rex-1, a gene encoding a transcription factor expressed in the early embryo, is regulated via Oct3/4 and Oct-6 binding to an octamer site and a novel protein, Rox-1, binding to an adjacent site. 1998; Mol. Cell. Biol;18:1866-1878

Chinswangwatanakul W, Lewis J L, Manning M. Use of G418 resistance to select cells retrovirally transduced with the NeoR gene. Exp. Hematol. 1998; 26:1856

Dilber M S and Gahrton G. Suicide gene therapy: possible applications in haematopoietic disorders. Journal of Internal Medicine 2001, 249:359-367.

Franklin T J. Resistance of Escherichia coli to tetracyclines. Changes in permeability to tetracyclines in Escherichia coli bearing transferable resistance factors. Biochem. J. 1967;105:371.

Eiges R, Schuldiner M, Drukker M, Yanuka O, Itskovitz-Eldor J, Benvenisty N. Establishment of human embryonic stem cell-transfected clones carrying a marker for undifferentiated cells. 2001 Curr Biol 11:514-8.

Hosler B A, Rogers M B, Kozak C A and Guda L J. An octomer motif contributes to the expression of the retinoic acid-regulated zinc finger gene Rex-1 (Zpf-42) in F9 teratocarcinoma cells. Mol. Cell. Biol. 1993;13:2919-2928

Karreman C. A new set of positive/negative selectable markers for mammalian cells. Gene Sep. 18, 1998;218(1-2):57-61

Lamb K, Rosfjord E, Brigman K, Rizzino A. Binding of transcription factors to widely-separated cis-regulatory elements of the murine FGF-4 gene. Mol. Reprod. Dev. August 1996;44(4):460-71

Le Grice S F J and Matzura H. Binding of RNA polymerase and the catabolite gene activator protein within the cat promoter of Escherichia coil J. Mol. Biol. 1981;150:185.

Maeda T. O-Wang J, Matsubara H, Asano T, Ochiai T, Sakiyama S, Tagawa M. A minimum c-erbB-2 promoter-mediated expression of herpes simplex virus thymidine kinase gene confers selective cytotoxicity of human breast cancer cells to ganciclovir. Cancer Gene Ther. November 2001; 8(11):890-6

Matz M V, Fradkov A F, Labas Y A. Fluorescent protein from nonbioluminescent Anthzoa species. Nat. Biotech. 1999; 17:969-973.

Reubinoff B E, Pera M F, Fong C Y, Trounson A, Bongso A. Embryonic stem cell lines from human blastocysts: somatic differentiation in vitro. Nat. Biotechnol. April 2000; 18(4):399-404.

Rogers M B, Hosler B A and Gudas L J. Specific expression of a retinoic acid-regulated, zinc-finger gene, Rex-1, in preimplantation embryos, trophoblast and spermatocytes. Development 1991; 113:815-824

Schöler H R Octamania:the POU factors in murine development. Trends Genet. 1991 ;7:323-329.

Shamblott M J, Axelman J, Wang S, Bugg E M, Littlefield J W, Donovan P J, Blumenthal P D, Huggins G R, Gearhart J D. Derivation of pluripotent stem cells from cultured human primordial germ cells. Proc Natl Acad Sci U S A. Nov. 10, 1998;95(23):13726-31.

Sykes R B and Matthew M. The beta-lactamase of gram-negative bacteria and their roles in resistance to beta-lactam antibiotics. J. Antimicrob. Chemother. 1976;2: 115

Thomson J A, Itskovitz-Eldor J, Shapiro S S, Waknitz M A, Swiergiel J J, Marshall V S, Jones J M. Embryonic stem cell lines derived from human blastocysts. Science. Nov. 6, 1998;282(5391):1145-7.

Yuan H, Corbi N, Basilico C, Dailey L. Developmental-specific activity of the FGF-4 enhancer requires the synergistic action of Sox2 and Oct-3. Genes Dev. Nov. 1, 1995;9 (21):2635-45

Altschul, S. F. et al (1990). Basic local alignment search tool. J. Mol. Biol., 215, 403-410

Andrews P. W. et al (1984c). Two monoclonal antibodies recognizing determinants on human embryonal carcinoma cells react specifically with the liver isozyme of human alkaline phosphatase. Hybridoma, 3, 33-39.

Andrews, P. W. (1984) Retinoic acid induces neuronal differentiation of a cloned human embryonal carcinoma cell line in vitro. Dev. Biol., 103, 285-293.

Andrews, P. W. et al (1984a). Three monoclonal antibodies defining distinct differentiation antigens associated with different high molecular weight polypeptides on the surface of human embryonal carcinoma cells. Hybridoma, 3, 347-361.

Andrews, P. W. et al (1987). Cell lines from human germ cell lines. In: Teratocarcinomas and embryonic stem cells: A practical approach (E. J. Robertson, ed.). IRL Press, Oxford, pp 207-248.

Ben-Shushan, E. et al (1998). Rex-1, a gene encoding a transcription factor expressed in the early embryo, is regulated via Oct-3/4 and Oct-6 binding to an octamer site and a novel protein, Rox-1 binding to an adjacent site. Mol Cell Biol, 18,1866-1878.

Duran, C. et al (2001). Hybrids of pluripotent and nullipotent human embryonal carcinoma cells: partial retention of a pluripotent phenotype. Int J Cancer, 93, 324-332.

Draper, J. S., Pigott, C., Thomson, J. A., Andrews, P. W. 2002 Surface antigens of human embryonic stem cells: changes upon differentiation in culture. J. Anatomy. In press

Fenderson, B. A. et al (1987). Glycolipid core structure switching from globo- to lacto- and ganglio-series during retinoic acid-induced differentiation of TERA-2-derived human embryonal carcinoma cells. Dev. Biol., 122, 21-34.

Kannagi, R. et al (1983). Stage-specific embryonic antigens (SSEA-3 and -4) are epitopes of a unique globo-series ganglioside isolated from human teratocarcinoma cells. EMBO J., 2, 2355-2361.

McKenzie J. L., and Fabre, J. W. (1981). Human thy-1: unusual localization and possible functional significance in lymphoid tissues, J Immunol., 126, 843-850.

Schuldiner, M. et al (2000). Effects of eight growth factors on the differentiation of cells derived from human embryonic stem cells. Proc. Nat. Acad. Sci. (USA), 97, 11307-11312.

Shevinsky, L. et al (1982). Monoclonal antibody to murine embryos defines a stage specific embryonic antigen expressed on mouse embryos and human teratocarcinoma cells. Cell, 30, 697-705.

Solter, D., and Knowles, B. B. (1978). Monoclonal antibody defining a stage-specific mouse embryonic antigen (SSEA-1). Proc. Nat. Acad. Sci. (USA,. 75, 5565-5569.

Thomson, J. A. et al (1998). Embryonic stem cell lines derived from human blastocysts. Science, 282, 1145-1147.

Williams, B. P. et al (1988). Biochemical and genetic analysis of the OKa blood group antigen. Immunogenetics, 27, 322-329.

TABLE 1

| Gene | Prodrug | Active drug |
|---|---|---|
| Thymidine kinase (HSVtk) | Ganciclovir | Ganciclovir |
| Triphosphate cytosine deaminase (CD) | 5-Fluorocytosine (5-FC) | 5-Fluorouracil (5-FU) |
| Guanosine-xanthine phosphoribosyl | 6-Thioguanine (6-Tg) | 6-Tg triphosphate |
| Transferase (gpt) | | |
| Purine nucleoside | 6-MeP-dR | 6-Methylpurine |
| phosphorylase | | |
| Nitroreductase (NTR) | CB1954 | Hydroxylamine |
| | | alkylating agents |
| CYP 2B1 (cytochrome P450) | Cyclophosphamide | 4-hydroperoxycyclo- |
| | | phosphamide |
| CYP 4B1 (cytochome P450) | 2-Aminoanthracene (2-AA) | DNA-alkylating |
| | | Agents |
| Cytochrome P450 | 4-Ipomeanol | Alkylating metabolites |
| Varicella zoster virus thymidine | AraM | AraM-MP |
| Kinase (VZVtk) | | |
| β-glucosidase | Amygdalin | Cyanide |
| β-lactamase | Vinca cephaloid | Vinca alk |
| b-glucoronidase | Phenolmustard glucoronide | Phenolmustard |
| Carboxylesterase | Topoisomerase 1 inhibitors | SN-38 |
| Alkaline phosphatase | Phenolmustard phosphate | Phenolmustard |
| | Etoposide phosphate | Etoposide |
| Carboxypeptidase G2 | Benroic acid mustard | Benzoic acid mustards |
| Glutaminases | | |

TABLE 2
PCR PRIMERS USED TO DETECT GENE EXPRESSION IN HUMAN ES CELLS

| | Accession | | | Product | Anneal | Product |
|---|---|---|---|---|---|---|
| Gene | No. | Sense (5′-3′) | Antisense (5′-3′) | position | Temp ° C. | size (bp) |
| Oct4 | NM_00270 | CGACCATCTGCCGCTTTGAG | CCCCCTGTCCCCCATTCCTA | 361-937 | 60 | 577 |
| Sox2 | Z31560 | CCCCCGGCGGCAATAGCA | TCGGCGCCGGGGAGATACAT | 410-857 | 60 | 448 |
| FGF4 | NM_002007 | CTACAACGCCTACGAGTCCTACA | GTTGCACCAGAAAAGTCAGAGTTG | 814-1183 | 55 | 370 |
| Rex1 | AF450454 | GCGTACGCAAATTAAAGTCCAGA | CAGCATCCTAAACAGCTCGCAGAAT | 793-488 | 56 | 306 |
| AFP | NM_001134 | AGAACCTGTCACAAGCTGTG | GACAGCAAGCTGAGGATGTC | 473-1148 | 57 | 676 |
| HBZ | MZ4173 | CTGACCAAGACTGAGAGGAC | ATGTCGTCGATGCTCTTCAC | 36-259 | 61 | 224 |
| HCG | NM_000737 | CAGGGGACGCACCAAGGATG | GTGGGAGGATCGGGGTGTCC | 349-858 | 62 | 510 |
| NeuroD1 | NM_002500 | AAGCCATGAACGCAGAGGAGGACT | AGCTGTCCATGGTACCGTAA | 240-818 | 66 | 579 |
| Sox1 | Y13436 | CTCACTTTCCTCCGCGTTGCTTCC | TGCCCTGGTCTTTGTCCTTCATCC | 1588-2435 | 58 | 848 |
| β-Actin | NM_001101 | ATCTGGCACCACACCTTCTACAATG | CGTCATACTCCTGCTTGCTGATCCAC | 326-1163 | 60 | 838 |
| | | AGCTGCG | ATCTGC | | | |

1. A method to manipulate the phenotype of a stem cell comprising;
providing a cell which has been transfected with a nucleic acid molecule wherein said nucleic acid molecule includes a promoter comprising at least one nucleic acid sequence motif which confers substantially stem cell specific expression on at least one selectable marker gene; and
providing conditions conducive to the proliferation of said cell.

2. The method according to claim 1 wherein said conditions are in vitro cell culture conditions.

3. The method according to claim 1 wherein said promoter is a synthetic promoter.

4. The method of claim 1 wherein said motif binds transcription factors of POU domain family.

5. The method according to claim 4 wherein said motif binds transcription factor Oct-3/4.

6. The method according to claim 5 wherein said motif binds an alternatively spliced form of Oct-3/4 selected from the group consisting of Oct-5 or -6.

7. The method of claim 1 wherein said motif is derived from an FGF-4 promoter.

8. The method according to claim 7 wherein said FGF-4 promoter is of human origin.

9. The method of claim 1 wherein said motif is at least one copy of CTTTGTT (SEQ ID NO: 30) and ATGCAAAT (SEQ ID NO: 31).

10. The method of claim 1 wherein said motif is at least one copy of CTTTGTT (SEQ ID NO: 30) and ATGCTAAT (SEQ ID NO: 33).

11. The method according to claim 9 wherein said motif comprises at least one copy of CTTTGTTXATGCAAAT (SEQ ID NO: 34) wherein X is at least one nucleotide base which serves as a linking molecule.

12. The method according to claim 10 wherein said motif comprises at least one copy of CTTTGTTXATGCTAAT (SEQ ID NO: 35) wherein X is at least one nucleotide base which serves as a linking molecule.

13. The method according to claim 9, wherein said motif is CTTTGTTcgaATGCAAAT (SEQ ID NO: 36).

14. The method of claim 1, wherein said nucleic acid molecule comprises at least 2 motifs.

15. The method of claim 1, wherein said nucleic acid molecule comprises a plurality of motifs.

16. The method according to claim 15 wherein said nucleic acid molecule comprises at least 3, 4, 5, 6, 7, 8, 9 or 10 motifs.

17. The method of claim 1, wherein said motif is derived from aRex-1 (zfp-42) promoter.

18. The method according to claim 17 wherein said Rex-1 promoter is of human origin.

19. The method according to claim 17 wherein said Rex-1 promoter is of murine origin.

20. The method of claim 17, wherein said Rex-1 promoter is ATTTGCAT (SEQ ID NO: 37).

21. The method of claim 1, wherein said stem cells are haemopoietic stem cells; neural stem cells; bone stem cells; muscle stem cells; mesenchymal stem cells; trophoblastic stem cells; epithelial stem cells; endodermal stem cells; embryonic stem (ES) cells; or embryonal germ (EG) cells.

22. The method according to claim 21 wherein said epithelial stem cells are derived from gastrointestinal mucosa, kidney, bladder, mammary gland, uterus, prostate, or an endocrine gland.

23. The method of claim 22, wherein said endocrine gland is a pituitary gland.

24. The method according to claim 21 wherein said endodermal stem cells are derived from liver, pancreas, lung or blood vessels.

25. The method of claim 21 wherein said stem cells are primate stem cells.

26. The method according to claim 25 wherein said stem cells are human stem cells.

27. The method of claim 21, wherein said embryonic stem cells are embryonal carcinoma cells.

28. The method according to claim 27 wherein said embryonal carcinoma cells are TERA2 cells.

29. The method according to claim 28 wherein said embryonal carcinoma cells are NTERA 2 cells.

30. The method of claim 1, wherein said selectable marker a gene encodes a polypeptide capable of fluorescence emission when excited by light.

31. The method according to claim 30 wherein said polypeptide is selected from the group consisting of BFP, CFP, YFP, GFP, RFP and variants thereof.

32. The method of claim 1, wherein said selectable marker gene encodes a polypeptide which confers resistance to an antibiotic.

33. An isolated nucleic acid molecule comprising a promoter of a gene, wherein said promoter comprises a motif, and wherein said motif, has substantially stem cell specific expression and which is operably linked to at least one selectable marker, wherein said motif is bound by a transcription factor of a POU family.

34. The isolated nucleic acid molecule according to claim 33 wherein said transcription factor is Oct-3/4.

35. The isolated nucleic acid molecule according to claim 34 wherein said transcription factor is Oct-5 and-6.

36. The isolated nucleic acid molecule of claim 33, wherein said motif is at least one copy of CTTTGTT (SEQ ID NO: 30) and ATGCAAAT (SEQ ID NO: 31).

37. The isolated nucleic acid molecule of claim 33, wherein said motif is at least one copy of CTTTGTT (SEQ ID NO: 30) and ATGCTAAT (SEQ ID NO: 33).

38. The isolated nucleic acid molecule according to claim 36 wherein said motif comprises at least one copy of CTTTGYTXATGCAAAT (SEQ ID NO: 34) wherein X is at least one nucleotide base which serves as a linking molecule.

39. The isolated nucleic acid molecule according to claim 37 wherein said motif comprises at least one copy of CTTTGTTXATGCTAAT (SEQ ID NO: 35) wherein X is at least one nucleotide base which serves as a linking molecule.

40. The isolated nucleic acid molecule according to claim 33 wherein said motif is derived from a Rex-1 (zfp-42) promoter.

41. The isolated nucleic acid molecule according to claim 40 wherein said Rex-1 (zfp-42) promoter is of human origin.

42. The isolated nucleic acid molecule according to claim 40 wherein said Rex-1 (zfp-42) promoter is of murine origin.

43. The isolated nucleic acid molecule of claim 33, wherein said selectable marker encodes a prodrug activating polypeptide.

44. The isolated nucleic acid molecule according to claim 43 wherein said prodrug activating polypeptide is thymidine kinase; triphosphate cytosine deaminase; 5 guanosine -xanthine phosphoribosyl; transferase; purine nucleoside phosphorylase; nitroreductase; CYP 2B1; CYP 4B1; Cytochrome P450; Varicella zoster virus thymidinekinase (VZVtk); β-glucosidase; β-lactamase; β-glucoronidase; carboxylesterase; alkaline phosphatase; or carboxypeptidase G2.

45. A vector comprising the isolated nucleic acid molecule of claim 33.

46. A stem cell transfected with the isolated nucleic acid molecule of claim 33.

47. The stem cell according to claim 46 wherein said stem cell is a haemopoietic stem cell; neural stem cell; bone stem cell; muscle stem cell; mesenchymal stem cell; trophoblastic stem cell; epithelial stem cell; endodermal stem cell; embryonic stem (ES) cell; or embryonal germ (EG) cell.

48. The stem cell according to claim 47 wherein said epithelial stem cells are derived from gastrointestinal mucosa, kidney, bladder, mammary gland, uterus, prostate, or an endocrine gland.

49. The stem cell according to claim 48 wherein said endocrine gland is a pituitary gland.

50. The stem cell according to claim 47 wherein said endodermal stem cells are derived from liver, pancreas, lung or blood vessels.

51. The stem cell of claim 47, wherein said stem cells are primate stem cells.

52. The stem cell according to claim 51 wherein said stem cells are human stem cells.

53. The stem cell according to claim 47 wherein said embryonic stem cells are embryonal carcinoma cells.

54. The stem cell according to claim 53 wherein said embryonal carcinoma cells are TERA2 cells.

55. The stem cell according to claim 54 wherein said embryonal carcinoma cells are NTERA 2 cells.

56. A differentiated cell transfected with the isolated nucleic acid molecule of claim 33.

57. The differentiated cell according to claim 56 wherein said differentiated cell is selected from the group consisting of: a nerve cell; a mesenchymal cell; a muscle cell; a liver cell; a kidney cell; a blood cell; a panceatic β cell; an epithelial cell; and an endothelial cell.

58. A cell culture comprising the transfected stem cell of claim 46.

59. A cell culture comprising the differentiated cell according to claim 56.

60. An organ/tissue comprising at least one differentiated cell according claim 56.

61. A method to ablate a differentiated cell which has or is de-differentiating to a stem cell or lineage restricted stem cell, comprising exposing said cell to an agent to which said cell has been sensitized.

62. A method according to claim 61 wherein said method is an in vivo method.

63. The method according to claim 61 wherein said method is an in vitro method.

64. The method of claim 61, wherein said agent is ganciclovir; 5-fluorouracil (5-FU); 6-Tg triphosphate; 6-methylpurine; hydroxylamine; 4-hydroperoxycyclo-phosphoamide; DNA-alkylating; alkylating metabolites; araM-MP; cyanide; vinca alk; phenolmustard; SN-38; phenolmustard; etoposide; or benzoic acid mustards.

65. The method of claim 61, wherein said agent is diptheria toxin or pseudomonas exotoxin.

66. The method of claim 1, further comprising maintaining or storing said cell.

67. The method of claim 32, wherein the polypeptide which confers resistance to an antibiotic is β lactamase, tetracycline resistance polypeptide, chloramphenicol acetyltransferase, aminoglycoside phosphotransferase, hygromycin phosphotransferase, puromycin N acetyltransferase, or variants thereof.


SEQUENCE LISTING (37 sequences)

SEQ ID NO: 1 - DNA, 166 bp, Mus musculus
cgcggggcaggcgagtaggagggggcgccggctatatataccactgctccggagggctgggcgcgcggggactatcccgccaccgttgcgtccctatttgctctcgctacttaggtctgtgcgcagcactcaccgaactcacggcccgcagctcgaactcacgcac

SEQ ID NO: 2 - DNA, 181 bp, Mus musculus
cgcggggcaggcgagtaggagggggcgccggctatatataccactgctccggagggctgggcgcgcggggactatcccgccaccgttgcgtccctatttgctctcgctacttaggtctgtgcgcagcactcaccgaactcacggcccgcagctcgaactcacgcacggcccgcgggccggg

SEQ ID NO: 3 - DNA, 185 bp, Homo sapiens
cgcgggagcgggcgagtaggagggggcgccgggctatatatatagcggctcggcctcgggcgggcctggcgctcagggaggcgcgcactgctcctcagagtcccagctccagccgcgcgctttccgcccggctcgccgctccatgcagccggggtagagcccggcgcccgggggccccgtcgctt

SEQ ID NO: 4 - DNA, 322 bp, Homo sapiens
cgcgggagcgggcgagtaggagggggcgccgggctatatatatagcggctcggcctcgggcgggcctggcgctcagggaggcgcgcactgctcctcagagtcccagctccagccgcgcgctttccgcccggctcgccgctccatgcagccggggtagagcccggcgcccgggggccccgtcgcttgcctcccgcacctcctcggttgcgcactcccgcccgaggtcggccgtgcgctcccgcgggccgccacaggcgcagctctgccccccagcttcccgggcgcactgaccgcctgaccgacgcacggccctcgggccggg

SEQ ID NO: 5 - DNA, 162 bp, herpes simplex
ccccgcccagcgtctagtcattggcgaattcgaacacgcagatgcagtcggggcggcgcggtcccaggtccacttcgcatattaaggtgacgcgtgtggcctcgaataccgagcgaccctgcagcgacccgcttaacagcgtcaacagcgtgccgcagatct

SEQ ID NO: 6 - DNA, 4278 bp, Artificial Sequence
  vector sequence
tagttattactagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtagatgcgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgccgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 7 - DNA, 127 bp, Homo Sapiens
agatcttcactttgttcgaatgcaaatcatctctttgttcgaatgcaaattcaggatcctcactttgttcgaatgcaaatcatctctttgttcgaatgcaaattcaaagcttatcactagtgaattc

SEQ ID NO: 8 - DNA, 162 bp, Herpes Simplex; identical to SEQ ID NO: 5

SEQ ID NO: 9 - DNA, 1111 bp, Artificial Sequence
  IRES
gttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 10 - DNA, 20 bp, Homo sapiens
cgaccatctgccgctttgag

SEQ ID NO: 11 - DNA, 20 bp, Homo sapiens
ccccctgtcccccattccta

SEQ ID NO: 12 - DNA, 18 bp, Homo sapiens
cccccggcggcaatagca

SEQ ID NO: 13 - DNA, 20 bp, Homo sapiens
tcggcgccggggagatacat

SEQ ID NO: 14 - DNA, 23 bp, Homo sapiens
ctacaacgcctacgagtcctaca

SEQ ID NO: 15 - DNA, 24 bp, Homo sapiens
gttgcaccagaaaagtcagagttg

SEQ ID NO: 16 - DNA, 23 bp, Homo sapiens
gcgtacgcaaattaaagtccaga

SEQ ID NO: 17 - DNA, 25 bp, Homo sapiens
cagcatcctaaacagctcgcagaat

SEQ ID NO: 18 - DNA, 20 bp, Homo sapiens
agaacctgtcacaagctgtg

SEQ ID NO: 19 - DNA, 20 bp, Homo sapiens
gacagcaagctgaggatgtc

SEQ ID NO: 20 - DNA, 20 bp, Homo sapiens
ctgaccaagactgagaggac

SEQ ID NO: 21 - DNA, 20 bp, Homo sapiens
atgtcgtcgatgctcttcac

SEQ ID NO: 22 - DNA, 20 bp, Homo sapiens
caggggacgcaccaaggatg

SEQ ID NO: 23 - DNA, 20 bp, Homo sapiens
gtgggaggatcggggtgtcc

SEQ ID NO: 24 - DNA, 24 bp, Homo sapiens
aagccatgaacgcagaggaggact

SEQ ID NO: 25 - DNA, 20 bp, Homo sapiens
agctgtccatggtaccgtaa

SEQ ID NO: 26 - DNA, 24 bp, Homo sapiens
ctcactttcctccgcgttgcttcc

SEQ ID NO: 27 - DNA, 24 bp, Homo sapiens
tgccctggtctttgtccttcatcc

SEQ ID NO: 28 - DNA, 32 bp, Homo sapiens
atctggcaccacaccttctacaatgagctgcg

SEQ ID NO: 29 - DNA, 32 bp, Homo sapiens
cgtcatactcctgcttgctgatccacatctgc

SEQ ID NO: 30 - DNA, 7 bp, Homo sapiens
ctttgtt

SEQ ID NO: 31 - DNA, 8 bp, Homo sapiens
atgcaaat

SEQ ID NO: 32 - DNA, 7 bp, Homo sapiens
ctttgtt

SEQ ID NO: 33 - DNA, 8 bp, Homo sapiens
atgctaat

SEQ ID NO: 34 - DNA, 16 bp, Homo sapiens
  misc_feature (8)..(8): n is a, c, g, or t
ctttgttnatgcaaat

SEQ ID NO: 35 - DNA, 16 bp, Homo sapiens
  misc_feature (8)..(8): n is a, c, g, or t
ctttgttnatgctaat

SEQ ID NO: 36 - DNA, 18 bp, Homo sapiens
ctttgttcgaatgcaaat

SEQ ID NO: 37 - DNA, 8 bp, Mus musculus
atttgcat